(19) United States
(12) Patent Application Publication
(10) Pub. No.: US 2013/0338383 A1
(43) Pub. Date: Dec. 19, 2013

(54) PREGNANOLONE DERIVATIVES SUBSTITUTED IN 3ALPHA-POSITION WITH THE CATIONIC GROUP, METHOD OF THEIR PRODUCTION, USAGE AND PHARMACEUTICAL PREPARATION INVOLVING THEM

(71) Applicants: Hana Chodounska, Praha (CZ); Vojtech Kapras, Praha (CZ); Ladislav Vyklicky, Kamenice (CZ); Jirina Borovska, Tuklaty (CZ); Vojtech Vyklicky, Kamenice (CZ); Karel Vales, Zbraslav (CZ); Ales Stuchlik, Celakovice (CZ); Lukas Rambousek, Koprivnice (CZ)

(72) Inventors: Hana Chodounska, Praha (CZ); Vojtech Kapras, Praha (CZ); Ladislav Vyklicky, Kamenice (CZ); Jirina Borovska, Tuklaty (CZ); Vojtech Vyklicky, Kamenice (CZ); Karel Vales, Zbraslav (CZ); Ales Stuchlik, Celakovice (CZ); Lukas Rambousek, Koprivnice (CZ)

(73) Assignees: FYZIOLOGICKY USTAV AKADEMIE VED CESKE REPUBLICKY, V.V.I., Praha 4 (CZ); USTAV ORGANICKE CHEMIE A BIOCHEMIE AKADEMIE VED CESKE REPUBLIKY, V.V.I., Praha 6 (CZ)

(21) Appl. No.: 13/984,320

(22) PCT Filed: Feb. 15, 2012
(86) PCT No.: PCT/CZ2012/000016

§ 371 Date: Sep. 4, 2013

(30) Foreign Application Priority Data
Feb. 15, 2011 (CZ) PV 2011-81

(51) Int. Cl. C07J 41/00

(52) U.S. Cl. CPC C07J 41/0011; C07J 41/0005
USPC 552/521

(57) ABSTRACT

Pregnanolone derivatives, substituted in 3 alpha-position with the cationic group, of general formula I, and a method of the production of these compounds and their utilization for treatment of neuropsychiatric disorders related to imbalance of glutamatergic neurotransmitter system, such as ischemic damage of CNS, neurodegenerative changes and disorders of CNS, affective disorders, depression, post traumatic stress disorder, and other diseases related to stress, anxiety, schizophrenia, and psychotic disorders, pain, addictions, multiple sclerosis, epilepsy, and gliomas. The compounds are also used for production of veterinary and human pharmaceutical preparation for treatment of above mentioned diseases and for production of pharmaceutical preparations containing these compounds.

### FIELD OF THE INVENTION

This invention relates to cationic steroid compounds, methods of their production, their applications and pharmaceutical compositions containing them. The invention particularly deals with pregnanolone derivatives substituted in 3alpha-position with the group bearing the cation, bound in this position. These derivatives may be beneficial in treatment of central nervous system (CNS) diseases, especially ischemic CNS injury, neurodegenerative alterations and diseases, depression, post-traumatic stress disorder and other stress-related disorders, schizophrenia and various psychotic diseases, pain, addiction, multiple sclerosis and autoimmune disorders, epilepsy, and gliomas as well as other CNS tumors.

### BACKGROUND ART

Glutamate is the principal excitatory neurotransmitter in the central nervous system of mammals. During synaptic transmission, the post-synaptic responses occur via ionotropic and metabotropic glutamate receptors. Metabotropic receptors operate via G-proteins and mobilize calcium ions from intracellular compartments. Activation of ionotropic receptors results in increase in permeability of postsynaptic membrane for sodium, potassium and calcium cations by opening an ion channel, which is an integral part of the receptors.

Typical examples of ionotropic receptors are N-methyl D-aspartate (NMDA) receptors, AMPA and kainate receptors. Although current knowledge suggests specific role of various types of superfamily of glutamate receptors in the glutamate-induced excitotoxicity, ionotropic receptors are generally considered as key players in these processes. Activation of ionotropic receptors leads to alterations in intracellular concentrations of various ions, mainly of Na⁺ and Ca²⁺. Current research demonstrates that beside calcium, elevated intracellular levels of sodium ions can also lead to neuronal death. In neuronal cultures and in retina the activation of glutamate receptors may lead to damage even by sodium cation in absence of extracellular calcium ions. Nonetheless, toxicity of elevated glutamate levels is usually associated with elevations in intracellular concentrations of Ca²⁺. Currently it is well established that there is a direct relationship between excessive influx of calcium into cells and glutamate-induced damage to neurons. Glutamate-induced pathological calcium elevation is usually ascribed to prolonged activation of ionotropic receptors. Elevation in intracellular calcium then may trigger the down-stream neurotoxicity cascade, which involves uncoupling of mitochondrial electron transport from ATP production, supranormal activation of enzymes such as calpain and other proteases, induction of specific protein kinases, NO-synthase, calcineurins and endonucleases. These changes may also promote the production of toxic reactive molecules such as reactive oxygen species (ROS) and induce changes in cytoskeleton architecture and activation of signals leading to apoptosis and mitochondrial damage (Villmann and Becker, 2007).

A number of preclinical studies show a remarkable ability of NMDA receptor antagonists to prevent from the excessive exocytose of glutamate and damage to the CNS. From the clinical point of view; however, their therapeutic potential is rather limited. Regarding the fact that glutamate receptors are ones of the most abundant in the CNS, application of their antagonists leads to wide variety of side effects, ranging from motor impairment to induction of psychotic symptoms. On the contrary, a large divergence of NMDA receptors and differences in their distribution at synapses and at extrasynaptic sites offer a possibility to search for drugs which selectively influence only a limited subset of NMDA receptors and thus to avoid the induction of unexpected side effects, while retaining their therapeutic neuroprotective activity.

Previous results demonstrated that naturally occurring 3α5β-pregnanolone sulfate affects the activity of NMDA receptor by a use-dependent manner. Therefore, this molecule has a more pronounced inhibitory action on the tonically activated NMDA receptors than on those physically activated by glutamate during synaptic transmission. It was also demonstrated that activation of extrasynaptic tonically activated NMDA receptors is very important for excitotoxic action of glutamate (Petrovic et al., 2005).

Therefore, we have started the development and testing of novel NMDA receptor antagonists derived from neurosteroids. These newly synthesized drugs exhibit affinity to extrasynaptic NMDA receptors. Importantly, previous electrophysiological studies showed that these compounds bound preferentially to open NMDA receptor channels. Our compounds lack affinity for other types of receptor; so we could presume that they will not affect signal transmission between neurons. The suggested mechanisms of their action are the blockade of extrasynaptic tonically activated NMDA receptors and prevention of excessive action of glutamate on neurons.

In the last decade, the biomedical research focused on the study of the role of neurosteroids in the pathogenesis of number of neuropsychiatric diseases and evaluation of their therapeutic potential. Mechanisms of action of neurosteroids are conventionally associated with their activity on NMDA and GABA-A receptors. A number of experimental studies with animal models show their potential in therapy of several diseases of CNS, including neurodegenerative disorders, multiple sclerosis, affective disorders, alcoholism, pain, insomnia or schizophrenia (Morrow, 2007; Weaver, 2000).

Neurosteroids also play a crucial role in the regulation of reactivity to stress and stress-related CNS disorders. Corticosteroid levels are known that acutely increase after exposition to a stressor; this represents an adaptive mechanism. On the other hand, experimental models of chronic stress and depression in laboratory rodents show decreased levels of neurosteroids both in brain and in plasma. Similar findings are often reported in patients suffering from depressions and pre-menstruation syndrome suggesting impairments in the CNS homeostatic mechanisms in stress-related neuropsychiatric disorders.

Steroid compounds affect activity and plasticity of neural and glial cells during early life, and later in development they play an essential neuroprotective role in the adult CNS. Steroids are released by sexual and adrenal glands as well as in the CNS. Steroids secreted by peripheral glands reach brain, medulla and spinal cord via blood circulation. Nonetheless, some neural steroids (i.e., neurosteroids) are biosynthesized directly in the CNS. The most studied neurosteroids are pregnenolone, progesterone, dehydroepiandrosterone (DHEA), their reduced metabolites, and esters. Not much is known about regulation of neurosteroid synthesis in the CNS, but it is generally assumed that they may underlie interaction of multiple cell types in the CNS. Synthesis of progesterone by Schwann cells, surrounding peripheral nerves, is regulated by signals diffusing from neurons.

Neurotrophic and neuroprotective properties of some neurosteroids were convincingly demonstrated both in cultures and in vivo. Progesterone plays a pivotal role in neurological recovery from traumatic brain and spinal cord injury by mechanisms including protection against excitotoxic damage to the brain, lipid peroxidation and by induction expression of specific enzymes. For example, after cutting the spinal cord, this steroid increases the number of NO-synthase-expressing astrocytes in place adjacent to cut both in the distal and proximal segment of the cord.

This steroid was also shown to regulate formation of new myelin sheaths. This fact was shown in regenerating rat sciatic nerve in the culture with sensory neurons and Schwann cells. Progesterone also supports myelination by activation of genes coding for proteins participating in this process.

As mentioned before, neurosteroids importantly modulate the function of membrane receptors for various neurotransmitters, namely GABA_{A} receptors, NMDA receptors and sigmal-opioid receptors. These mechanisms are most likely responsible for psychopharmacological effects of steroids and may at least partly account for their anticonvulsant, anxiolytic, neuroprotective and sedation effects as well as for their influence upon learning and memory functions. For instance, pregnanolone sulfate was shown to be capable of reversing cognitive deficit in aged animals and exerting a protective effect on memory in several amnesia models. Recent studies have demonstrated direct effect of neurosteroids on intracellular receptors. Despite absence of direct evidence for binding of neurosteroids to corticoid receptors, they may obviously modulate their function indirectly, by interaction with protein kinases C and A, MAP-kinase (MAPK) or CaMKII. Moreover, pregnanolone and pregnanolone sulfate were shown to affect microtubule-associated proteins and increase the rate of microtubule polymeration, which may in turn affect neuronal plasticity.

Sulfated esters of neurosteroids also play a physiological role in the regulation of receptors for excitatory and inhibitory neurotransmitters and participate in the natural protective properties of CNS tissue. Sulfated esters of neurosteroids and their analogs are promising molecules, potentially beneficial for treatment of CNS disorders. Nonetheless, a ratio between neurosteroids and their sulfated esters is maintained enzymatically in the CNS tissue in vivo. Exogenous administration of sulfated esters may not lead to improvement in the protective functions, due to enzyme activity. The invented molecules are metabolically more stable analogs of sulfated esters of neurosteroids. Sulfated and thus polar steroids compounds generally do not penetrate the blood-brain, but it was demonstrated that intravenously administered pregnanolone sulfate reach the brain (Wang et. al., 1997). However, the ratio of sulfated and free steroid in the brain is stable. The transport of sulphated analogs is probably mediated by active exchange mechanisms associated with so-called organic anion transport protein (OATP), which is expressed in the cells of brain tissue.

Advantage of our molecules is that they retain similar pharmacological and physiological properties as pregnanolone sulfate, but they are not metabolically deactivated by sulfatases into non-conjugated metabolites.

### DESCRIPTION OF THE INVENTION

The present invention relates to compounds of general formula I,

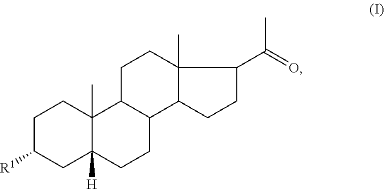

in which R¹ represents the group of general formula R³—R²—C(R¹³)—R¹⁴—, where R² means (CHₘ)ₙ₋ group, wherein m is 0 to 2, n is 1 to 18 and forms straight or a branching chain, which may be additionally substituted by one or more halogen atoms or primary, secondary or terciary amino group, which may be either free or in the case of primary amino group protected by a removable protecting group, chosen from tert.butoxycarbonyl, trityl, benzyloxycarbonyl, 9-fluorenylmethoxycarbonyl, or p-nitrobenzyloxycarbonyl,

R³ represents cationic group, chosen from guanidyl group of general formula (a)

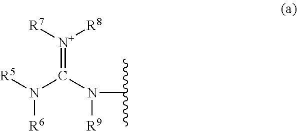

or ammonium group of general formula (b)

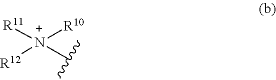

where R⁵-R¹² are hydrogen atoms or alkyl or alkenyl groups with 1 to 18 carbon atoms in a straight or a branching carbon chain, R¹³ is chosen from a group, involving oxygen, nitrogen or sulfur atom bound by a double bond to carbon, or R¹³ are two hydrogen atoms; R⁴ is bivalent or multivalent atom, chosen from oxygen, nitrogen or carbon atom and in case where R⁴ is multivalent atom, that is carbon or nitrogen, its additional valent(s) is (are) hydrogen(s) and any of hydrogen atoms may be substituted by alkyl or alkenyl having from 1 to 4 carbon atoms.

In one aspect of the invention pregnanolone derivatives, substituted in the 3alpha-position with the cationic group are of general formula I described above, where the substiuent —R²—C(R¹³)—R¹⁴— is missing, R¹═R³=ammonium group of general formula (b) described above and R¹⁰-R¹² of this group are hydrogen atoms or alkyl or alkenyl groups with 1 to 18 carbon atoms in a straight or a branched carbon chain.

Another object of the present invention is the method of preparation of compounds of general formula I, where R¹ is as given above, and the substituent R³ is guanidyl group of the formula (a) and R⁴ means oxygen atom. The method is based on the process where the reaction mixture, containing 3alpha-hydroxy-5beta-pregnane-20-one of formula II

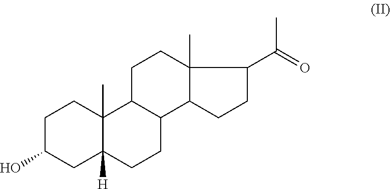

and arginine protected by suitable protecting group chosen from tosyl, 2,2,5,7,8-pentamethyl-6-sulfonyl (Pmc), 2,2,4,6,7-pentamethyl-dihydrobenzofuran-5-sulfonyl-(Pbf), mesityl-2-sulfonyl (Mts), 4-methoxy-2,3,6-trimethylphenylsulfonyl (Mtr), 1,2-dimethylindole-3-sulfonyl (MIS), ω,ω′-bis-tert-butyloxycarbonyl (bis-Boc), co-nitro, trifluoroacetyl (tfa), ω,ω′-bis-benzyloxycarbonyl (bis-Z), or ω,ω′-bis-allyloxycarbonyl (bis-Alloc) groups, is dissolved in suitable dry solvent, chosen from chloroform, dichloromethane, benzene, toluene, ethylacetate, or acetonitrile under the inert atmosphere. The reaction mixture is then cooled in ice bath. Condensing agents, which is dicyclohexylcarbodiimide or 1-(3-dimethylamino-propyl)-3-ethylcarbodiimide and a catalytic agent dimethylaminopyridine, dissolved in convenient solvent chosen from benzene or toluene, are added dropwise to the stirred mixture. The reaction mixture is prevented against the air humidity and stirred 10-48 hours at temperatures between 0 and 50° C. Then is poured into saturated water solution of sodium or potassium bicarbonate and the product is extracted with an organic solvent, in which is well soluble. Collected organic phases are then washed with saturated water solution of sodium chloride to remove sodium bicarbonate. The extract is dried over magnesium sulfate or sodium sulfate and the solvent is evaporated preferably by distillation under vacuum. The crude material is triturated with minimal amount of acetone and precipitated dicyclohexylurea is filtered off to obtain the compound of general formula I. The product obtained is purified, where appropriate. In case that arginine bears protecting groups, their removing is implemented so that the obtained compound is dissolved in a mixture of carboxylic acid and alcohol. To this solution a hydrogenation catalyst is added, preferably Pd/C or platinum black. After the hydrogenating reaction is completed in 48-72 h, the catalyst is filtered off, and the solvent is evaporated.

One of preferred embodiments of presented invention consist in that the reaction mixture is mixed from 10 to 12 h, ethylacetate is used as organic solvent, the product is purified by crystallization, or by chromatography on the silica gel column. Protecting group is removed in the mixture of acetic acid and methanol. The time of hydrogenation is preferably 72 hours.

In one aspect of the method according to presented invention protecting group of arginine structure of the compound of general formula I obtained as described above, benzyloxycarbonyl- or (2,2,4,6,7-pentamethyl-dihydrobenzofuran-5-sulfonyl)-(Pbf) group, is removed by trifluoroacetic acid treatment of the protected derivative. The reaction mixture is allowed to react from 16 to 72 hours at temperatures from 0° to 50° C. Then the mixture is poured into saturated solution of sodium or potassium bicarbonate, and the product is extracted with an organic solvent, in which is well soluble, chosen from chloroform, dichloromethane, or dichloroethane. Collected organic phases are washed with 5% aqueous hydrochloric acid. The extract is dried over drying agent and the solvent is evaporated. The crude material is purified, where appropriate, e.g. by crystallization to afford the dihydrochloride of compound of general formula I.

In next aspect of the method according to presented invention the reaction mixture is allowed to react under the room temperature, the bicarbonate used is sodium bicarbonate, drying agent is magnesium or sodium sulfate, and the solvent is preferably evaporated by distillation under the reduced pressure.

The following object of invention is also the method of the preparation of compound of general formula I, where R¹ means as given above and R³ is quaternary ammonium salt of general formula (b)

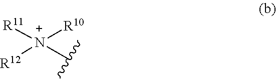

with the various length of chains connecting the amino-group with carboxyl creating ester bond with the compound of general formula II. The method is based on that the appropriate quaternary salt of co-carboxylic acid is suspended in anhydrous dichloromethane under inert atmosphere. Into the reaction mixture of the appropriate temperature from −50 to +20 deg C. is added suitable chlorinating agent, chosen from a group consisting of thionylchloride, phosphorus oxychloride, oxalyldichloride. The reaction can be catalyzed by convenient catalyst. Reaction mixture is then stirred for the appropriate reaction time 8-72 h to dissolve all solids, volatile components are then evaporated in vacuo and crude product is dissolved in the mixture of dry nitromethane and pyridine under the inert atmosphere. The compound of general formula II is added and the mixture is then stirred for 2-24 h until reaction is quenched with water, then acidified to pH 4 with 5% aqueous solution of hydrochloric acid, organic components are extracted into chloroform and this is washed with saturated solution of sodium chloride. Obtained solution is dried over drying agent and the solvent then evaporated. Trituration with benzene removes the unreacted starting material. The remaining product is purified if appropriate, e.g. by crystallization from the mixture of suitable solvents, to give the product of general formula I, where R³ means as given in general formula (b), i.e. R³ represents the quaternary ammonium salt.

In next aspect of presented invention the temperature of the reaction mixture is preferably 0 deg C., as the chlorinating agent is preferably used oxalyldichloride and as the catalyst dimethylformamide, at the same time the reaction mixture is stirred at first 16 h and after the addition of the compound of general formula II next 4 hours. Drying agent is magnesium or sodium sulfate, solvent is evaporated by distillation under the vacuum and the product is purified by crystallization from the mixture of chloroform and n-heptane.

Presented invention also includes pregnanolone derivatives substituted in position 3alpha with cationic group of general formula I, for usage for treatment of neuropsychiatric disorders related to dysbalances of glutamatergic neurotransmitter system, as ischemic CNS injury, neurodegenerative changes, and disorders of CNS, mood disorders, depression, post-traumatic stress disorder, and other stress-related disorders, anxiety, schizophrenia and other psychotic illnesses, pain, addiction, multiple sclerosis, epilepsy, and gliomas.

The object of invention is also the use of compounds of general formula I for production of veterinary and human pharmaceutical preparations for treatment of neuropsychiatric disorders related to imbalance of glutamatergic neurotransmitter system, ischemic damage of CNS, neurodegenerative changes and disorders of CNS, affective disorders, depression, PTSD, and other diseases related to stress, anxiety, schizophrenia, and psychotic disorders, pain, addictions, multiple sclerosis, epilepsy, and gliomas.

The object of invention is also pharmaceutical preparation containing pregnanolone derivatives substituted in position 3alpha with cationic group of general formula I as active component.

The object of invention is pharmaceutical preparation containing as active substance the pregnanolone derivatives substituted in position 3alpha with cationic group of general formula I, for use in the treatment of neuropsychiatric disorders related to dysbalance of glutamatergic neurotransmitter system as ischemic CNS injury, neurodegenerative changes, and disorders of CNS, mood disorders, depression, post-traumatic stress disorder, and other stress-related disorders, anxiety, schizophrenia, and other psychotic illnesses, pain, addiction, multiple sclerosis, epilepsy and, gliomas.

The object of invention is finally pharmaceutical preparation containing as active substance the pregnanolone derivatives substituted in position 3alpha with cationic group of general formula I for production of standards of neuroactive steroids utilized in experimental research, analytic chemistry. Moreover also utilization of these compounds as active substances contained in dietary supplements or cosmetic preparations designated for improvement of reactiones of particular parts of organisms on higher stress, namely oxidative, nutrient, or caused by free radicals, or by ageing.

The invention is based on the results of experiments, in which we studied the activity of pregnanolone sulfate on native and recombinant NMDA receptors. These experiments showed that the naturally occurring neurosteroid inhibits responses elicited by exogenous application of specific agonists of NMDA receptors. We have proved, that pregnanolone sulfate binds only to activate receptors (use-dependent activity) but does not bind to the ionic channel as some substances of Mg²⁺, ketamine, dizocilpine or memantine type. The rate of binding and the mechanism of action pregnanolone sulfate causes higher inhibitory effect on glutamate tonically activated receptors, than on physically activated receptors during the synaptic transmission. It was newly discover that analogues synthesized by us, which are the object of the invention, shows the same mechanism of action on the NMDA receptors.

Application of pregnanolone sulfate does not cause improvement of function studied as consequence of enzymatic activity, but molecules presented here constitute better analogues, which are not hydrolysable by sulfatases.

Various structural modifications of our invented compounds of general formula I have shown only minimal differences in their biological activity; these findings are congruent with previous electrophysiological results using patch-clamp technique and assessing binding kinetics of these compounds on the NMDA receptors.

The data confirm capability of NMDA receptor antagonists to prevent the excessive release of glutamate and subsequent damage of the CNS leading to deterioration of behavior. From the clinical point of view; however, their therapeutic potential is rather limited regarding the fact that their application leads to wide variety of side effects, ranging from motor impairment to induction of psychotic symptoms.

Main advantage of 3αC-substituted analogs of pregnanolone, use-dependent NMDA antagonists, constitutes absence of serious side effect typical for competitive NMDA antagonists, while retaining their therapeutic activities.

### DESCRIPTION OF FIGURES ON DRAWINGS

FIG. 1A presents current response elicited by 1 mmol·l⁻¹ of glutamic acid and reversible inhibition of the current response caused by the compound from the example 2, which was applied in the concentration 10 μmol·l⁻¹ simultaneously with the glutamic acid in time intervals presented by empty rectangles. The membrane potentials were maintained at −60 mV or +60 mV. Patch-clamp technique was used to record the currents from cultivated HEK293 cells with transfected NR1/NR2B NMDA receptors. Relative inhibition elicited by the compound from the example 2 was calculated from the formula (1−a/b)*100(%).

FIG. 1B is the diagram showing an independence of average inhibition elicited by the compound from the example 2 (10 μmol·l⁻¹) in time intervals presented by empty rectangles depicted on the axis y, on the maintained membrane potential depicted on the axis x.

FIG. 2 presents an effect of pregnanolone arginate on spontaneous locomotor activity in the open-field test. Total path length in metres is depicted on the axis y, first column responds to intact animals, middle column responds to control animals injected with saline and cyclodextrin, third column responds to animals injected with pregnanolone argininate.

FIG. 3 presents an effect of pregnanolone arginate and pregnanolone nitrate on locomotion and cognitive function in Carousel maze. The total distance (m) per session in the AAPA training is depicted on the axis y. Control animals obtained cyklodextrine, dizocilpine (MK) was used as a positive control.

FIG. 4 presents a number of entrances into shock sector, related to cognitive functions of animals influenced by of pregnanolone arginate and/or pregnanolone nitrate. The number of entrances is plotted on the axis y. Control animals obtained cyklodextrine, dizocilpine (MK) was used as a positive control; * denotes p<0.05, *** denotes p<0.001 compared to cyclodextrin controls.

### EXAMPLES

### List of Abbreviations

DMSO dimethylsulfoxide
DMAP 4-dimethylaminopyridine
DCC dicyklohexylkarbodiimid
DMF dimethylformamide
HRMS high resolution mass spectrometry
Boc tert-butoxycarbonyl
Pbf 2,2,4,6,7-pentamethyl-2,3-dihydrobenzofuran-5-sulfonyl
m multiplet
bm broad multiplet
d dublet
t triplet
EI electron impact ionozation
ESI electrospray ionization
eq. equivalent
IR infrared spectroscopy
MS mass spectroscopy
NMR nuclear magnetic resonance
Et ethyl
t-Bu tertiary butyl
Ac acetyl
HEK human embryonic kidney cells
GFP green fluorescent protein
IC₅₀ the half maximal inhibitory concentration
Opti-MEM® 1 minimum essential media, Invitrogen's product
DHEA 5-dehydroepiandrosterone
EGTA ethylene glycol tetraacetic acid
EDTA ethylene diamine tetraacetic acid
HEPES 4-(2-hydroxyethyl)-1-piperazineethanesulfonic acid

### Biological Activity on Cell Cultures

Degree of activated NMDA receptor inhibition by steroid cationic compounds was measured in vitro electrophysiologically on cultivated HEK293 cells (Human Embryonic Kidney 293 cells) 24-48 h after the transfection with DNA plasmids, coding NR1-1a and NR2B subunit of NMDA receptor. Transfected cells were identified by means of fluorescent green protein (GFP) fluorescence. Its genus was transfected together with the both receptor subunit genes.

Steroid-containing solutions were prepared from fresh solution (20 mmol·l⁻¹, of steroid dissolved in dimethyl-sulfoxide, DMSO), which was added to the extracellular solution containing 1 mmol·l⁻¹ glutamic acid and 10 μmol·l⁻¹ of glycine. Same concentrations of DMSO were added to all other extracellular solutions.

Current responses produced by extracellular application of glutamic acid solution (1 mmol·l⁻¹) were measured from the whole cell by patch-clamp technique, which is used for the study of transport of charged particles through model and also natural biological membranes. The currents were measured at membrane potential maintained at −60 mV and +60 mV. Steroid compounds studied lowered response amplitude elicited by glutamic acid. Application of 10 μmol·l⁻¹ steroid solution the mean inhibition effect reached 65-70%. It can be compared with 100 μmol·l⁻¹ of endogenous neurosteroid 5β-pregnanolon-3α-yl-sulfate, which inhibited responses elicited by NMDA receptor to 67%.

### Example 1

### 20-Oxo-5β-pregnan-3α-yl (2S)-2-(benzyloxycarbonylamino)-5-(3-nitroguanidino)pentanoate

An oven-dried 100 mL flask with magnetic stir bar was charged with the mixture of compound II (300 mg, 0.94 mmol), N_{α}-(carbonyloxybenzyl)-N_{ω}-nitro-L-arginine (366 mg, 1.03 mmol) and DMAP (12 mg; 0.09 mmol). 20 mL of dry acetonitrile were added under the argon atmosphere and resulted mixture was cooled in ice bath. 1 mol·l⁻¹ solution of DCC in benzene (1.41 mL; 1.41 mmol) was then added dropwise to the stirred cold mixture. The cooling bath was then removed and the reaction mixture was stirred for 16 hrs. The reaction as quenched with saturated solution of NaHCO₃ (50 mL), extracted with EtOAc (3×25 mL), washed with brine (50 mL), dried with anhydrous MgSO₄, filtered and evaporated in vacuo. N,N′-Dicyclohexylurea was crystallized from minute amount of acetone and filtered off. Filtrate was concentrated in vacuo again and loaded in toluene on a column of silica (30 g). Elution with a mixture of hexanes:acetone (7:3) and subsequent evaporation of solvents gave 20-oxo-5β-pregnan-3α-yl (2S)-2-(benzyloxycarbonylamino)-5-(3-nitroguanidino)pentanoate as white foam (473 mg; 77%).

¹H NMR (500 MHz, CDCl₃) δ 7.41-7.31 m (5H, Ph), 5.67 (d, 1H, J=8.0 Hz, NHCbz), 5.12 (s, 2H, CH₂-Ph), 4.84-4.74 (m, 1H, 3-CH), 4.37-4.28 (m, 1H, 2′-CH), 3.63-3.52 (m, 1H, 5′ a-CH), 3.35-3.23 (m, 1H, 5′ b-CH), 2.57 (t, 1H, J=9.0 Hz), 2.12 (s, 3H, 21-CH₃), 0.94 (s, 3H, 19-CH₃), 0.60 (s, 3H, 18-CH₃).

¹³C NMR (101 MHz, CDCl₃) δ 209.54, 171.34, 159.24, 135.94, 128.70, 128.52, 127.77, 76.34, 67.59, 63.80, 56.60, 52.27, 44.32, 41.87, 40.45, 40.22, 39.09, 35.80, 34.88, 34.61, 32.09, 31.67, 31.51, 26.88, 26.51, 26.25, 24.41, 24.33, 23.22, 22.93, 20.88, 13.42.

IR (CHCl₃): 3397 (NH), 1729 (C═O, ester), 1702 (C═O, ketone), 1626, 1606 (C═NH), 1515 (carbamate), 1387 (CH₃), 1348 (NO₂), 1291, 1277 (CO), 1232 (carbamate), cm⁻¹.

ESI m/z 654.1 (45%, [M+H]⁺), 676.3 (100%, [M+Na]⁺); HRMS-ESI m/z 654.3861 ([M+H]⁺, C35H52O7N5 requires 654.3861).

### Example 2

### 20-Oxo-5β-pregnan-3α-yl L-argininate diacetate salt

Compound from the example 1 (216 mg; 0.33 mmol) was dissolved in a mixture of acetic acid (0.5 mL) and methanol (9.5 mL). 10% Pd on carbon (44 mg) was added and reaction mixture was stirred at r.t. under atmospheric pressure of hydrogen gas for 72 hrs. Catalyst was filtered off through pad of diatomaceous earth and the solvents were evaporated. Chromatography on silica gel (4 g) in MeOH:AcOH:H₂O afforded 20-Oxo-5β-pregnan-3α-yl L-argininate diacetate salt as an off-white foam (187 mg; 95%).

¹H NMR (500 MHz, CDCl₃:d₄-MeOD, δ: 1) δ 4.82-4.74 m (m, 1H, 3-CH), 3.47 (t, 1H, J=6.4 Hz, 2′-CH), 3.15 (t, 2H, J=6.8 Hz, 5′-CH), 2.58 (t, 1H, J=8.8 Hz, 17-CH), 2.17 (s, 3H, 21-CH₃), 2.01 (s, 6H, —OOCCH₃), 0.96 (s, 3H, 19-CH₃), 0.61 (s, 3H, 18-CH₃).

¹³C NMR (101 MHz, d₄-MeOD) δ 212.41, 169.93, 78.62, 65.01, 58.03, 53.89, 45.55, 43.44, 41.95, 41.85, 40.38, 37.34, 36.11, 35.92, 33.36, 31.72, 28.94, 28.19, 27.75, 27.67, 25.80, 25.57, 24.07, 23.81, 22.13, 13.88.

IR (KBr): 3342, 3267, 3167 (NH₃⁺), 1726 (C═O, ester), 1699 (C═O, ketone+guanidinium), 1679, 1601, (guanidinium), 1551, 1408 (AcO⁻), 1387 (CH₃), 1364 (COCH₃), 1235, 1167 (CO), cm⁻¹.

ESI m/z 475.3 (100%, [M−2AcOH+H]⁺); HRMS-ESI m/z 475.3640 ([M−2Cl+H]⁺, C27H47O3N4 requires 475.3643).

### Example 3

### 20-Oxo-5β-pregnan-3α-yl 2-[(tert-butoxycarbonyl)amino]-5-(3-{(2,2,4,5,7-pentamethyl-2,3-dihydrobenzofuran-6-yl)sulfonyl)guanidino}pentanoate

In an oven-dried 100 mL flask with magnetic stir bar was charged with the compound II (500 mg, 1.57 mmol), Boc-L-Arg(Pbf)-OH (994 mg, 1.88 mmol), dimethylaminopyridine (DMAP, 21 mg; 0.16 mmol) and flushed with argon. Solids were dissolved in dry benzene (45 mL) and the reaction mixture was cooled in ice bath. 1 mol·l⁻¹ solution of dicyclohexylcarbodiimide (DCC) in benzene (1 mol·l⁻¹, 1.41 mL; 1.41 mmol) was then added dropwise. The reaction mixture was heated up to r.t. and stirred for 16 hrs. The reaction was quenched with saturated solution of NaHCO₃ (50 mL). Product was extracted with EtOAc (3×25 mL), washed with brine (50 mL), dried with anhydrous MgSO₄, filtered and evaporated in vacuo. N,N′-Dicyclohexylurea was crystallized from minute amount of acetone and filtered off. Filtrate was concentrated in vacuo again and loaded in toluene on a column of silica (50 g). Elution with hexanes:acetone (7:3) and subsequent evaporation of solvents gave 20-oxo-5β-pregnan-3α-yl 2-((tert-butoxycarbonyl)amino)-5-(3-((2,2,4,5,7-pentamethyl-2,3-dihydrobenzofuran-6-yl)sulfonyl)guanidino)pentanoate as white foam (1.29 g; 99%).

[α]_{D}=+53.0 (c 0.234);

¹H NMR (500 MHz, CDCl₃) δ 6.31 (bm, 1H, guanidine), 6.04 (s, 2H, guanidine), 5.30 (d, 1H, J=7.7 Hz, NHBoc), 4.82-4.74 (m, 1H, 3-CH), 4.25-4.19 (bm, 1H, 2′-CH), 3.40-3.30 (m, 1H, 5′ a-CH), 3.25-3.15 (m, 1H, 5′ b-CH), 2.96 (s, 2H, CH₂), 2.59 (s, 3H, CH₃), 2.53 (s, 3H, CH₃), 2.53 (t, 1H, J=9.0 Hz), 2.10 (s, 3H, 21-CH₃), 1.46 (s, 3H, 2xCH₃), 1.43 (s, 9H, tBu), 0.94 (s, 3H, 19-CH₃), 0.59 (s, 3H, 18-CH₃).

¹³C NMR (101 MHz, CDCl₃) δ 209.55, 171.81, 158.70, 156.01, 138.37, 133.08, 132.35, 124.54, 117.42, 86.31, 80.49, 75.94, 63.79, 56.57, 44.29, 43.29, 41.88, 40.89, 40.41, 39.10, 35.80, 34.92, 34.62, 32.13, 31.49, 28.59, 28.35, 26.89, 26.57, 26.26, 24.39, 23.23, 22.91, 20.87, 19.21, 17.84, 13.40, 12.45.

IR (CHCl₃): 3432, 3345 (NH), 1728 (C═O, ester), 1699 (C═O, ketone), 1633, 1623, 1559 (guanidin), 1506 (NHBoc), 1408 (guanidin), 1393 (tBu), 1385, 1370 (CH₃), 1358 (COCH₃), 1158 (SO₂), cm⁻¹.

ESI m/z 827.5 (63%, [M+H]⁺), 849.5 (100%, [M+Na]⁺); HRMS-ESI m/z 827.49907 ([M+H]⁺, C45H71O8N4S requires 827.49871).

For C45H70N4O8S (827.1) calculated: 65.34%; C, 8.53%; H, 6.67%; N, 3.88%; S, found: 65.51%; C, 8.68%; H, 6.43%; N, 3.70%; S.

### Example 4

### 20-Oxo-5β-pregnan-3α-yl L-argininate, dihydrochloride salt

Compound from the example 3 (430 mg; 0.52 mmol) was dissolved in neat trifluoroacetic acid (0.5 mL) and stirred for 48 hrs at r. t. The reaction mixture was then poured into saturated solution of NaHCO₃ (50 mL), extracted with chloroform (4×20 mL), washed with 5% solution of HCl (50 mL), dried with anhydrous Na₂SO₄, filtered and evaporated under the reduced pressure. Crystallization in chloroform afforded 20-oxo-5β-pregnan-3α-yl L-argininate dihydrochloride salt as white crystals (225 mg; 79%);

[α]_{D}=+71.4 (c 0.224; MeOH);

¹H NMR (500 MHz, d₄-MeOD) δ 4.92-4.88 m (1H, 3-CH), 4.84-4.74 (m, 1H, 3-CH), 4.06 (t, 1H, J=6.4 Hz, 2′-CH), 3.27 (t, 2H, J=6.8 Hz, 5′-CH), 2.64 (t, 1H, J=8.8 Hz, 17-CH), 2.12 (s, 3H, 21-CH₃), 0.99 (s, 3H, 19-CH₃), 0.61 (s, 3H, 18-CH₃).

¹³C NMR (101 MHz, d₄-MeOD) δ 212.41, 169.93, 78.62, 65.01, 58.03, 53.89, 45.55, 43.44, 41.95, 41.85, 40.38, 37.34, 36.11, 35.92, 33.36, 31.72, 28.94, 28.19, 27.75, 27.67, 25.80, 25.57, 24.07, 23.81, 22.13, 13.88.

IR (KBr): 2935 (NH₃⁺), 1744 (C═O, ester), 1706 (C═O, ketone), 1667, 1652, 1625 (guanidinium), 1385 (CH₃), 1358 (COCH₃), 1226, 1193 (CO), cm⁻¹.

ESI m/z 475.4 (100%, [M−2Cl+H]⁺); HRMS-ESI m/z 475.36398 ([M−2Cl+H]⁺, C27H47O3N4 requires 475.36427).

For C27H48Cl2N4O3 (547.6) calculated: 59.22%; C, 8.84%; H, 12.95%; Cl, 10.23%; N, found: 58.95%; C, 8.72%; H, 13.11%; Cl, 9.99% N.

### Example 5

### 20-Oxo-5β-pregnan-3α-yl 2-(methylguanidino) acetate hydrochloride

Anhydrous creatine (59 mg, 0.5 mmol] was dissolved in tetrahydrofuran (15 ml), then the compound II (160 mg, 0.5 mmol), anhydrous magnesium sulfate (2 g) and catalytic amount of sulfuric acid was added. The mixture was stirred 16 h at the room temperature. Then it was poured into the ice water and product extracted into the chloroform (4×20 ml), organic layer was washed by cold solution of sodium hydrogen carbonate (5%), diluted hydrochloric acid (5%, 20 ml), dried by anhydrous sodium sulfate. The mixture was filtrated and solvents evaporated under the reduced pressure. Crystallization from chloroform afforded 20-oxo-5β-pregnan-3α-yl2-(methylguanidino) acetate hydrochloride (107 mg; 47%).

¹H NMR (500 MHz, d₄-methanol) δ 4.92-4.88 m (1H, 3-CH), 4.79 (d, 1H, 3-CH), 2.57 (t, 1H, J=8.8 Hz, 17-CH), 2.12 (s, 3H, 21-CH₃), 0.99 (s, 3H, 19-CH₃), 0.61 (s, 3H, 18-CH₃).

IR (KBr): 2935 (NH₃⁺), 1744 (C═O, ester), 1706 (C═O, keton), 1667, 1652, 1625 (guanidinium), 1385 (CH₃), 1358 (COCH₃), 1226, 1193 (CO), cm⁻¹.

ESI m/z 418.3 (100%, [M−Cl+H]⁺); HRMS-ESI m/z 418.3073 ([M−Cl+H]⁺, for C₂₄H₄₀O₃N₃ calculated 418.3070).

### Example 6

### 20-Oxo-5β-pregnan-3α-yl 4-(trimethylammonium) butanoate chloride

3-Carboxy-N,N,N-trimethylpropan-1-ammonium chloride (prepared according to Lindstedt and Lindstedt, 1965, 69 mg; 0.38 mmol) was suspended in anhydrous CH₂Cl₂ (1 mL) under argon. The reaction flask was cooled in ice bath and oxalyl chloride (0.5 mL; 5.82 mmol) was added dropwise, followed by catalytic amount of dry DMF (3 μL; 0.03 mmol). The heterogeneous mixture was then brought to r.t. and stirred for 16 hrs, during which all the solids dissolved. The mixture was evaporated under the reduced pressure and solid residue was dissolved in dry nitromethane (2 mL) and dry pyridine (0.10 mL; 1.24 mmol) under argon. Compound II (100 mg; 0.31 mmol) was added to this reaction mixture, which was then stirred for 4 hrs. Reaction was quenched with water (10 mL) and acidified to pH 4 with 5% aq. HCl. Product was extracted with CHCl₃ (3×20 mL), solution was washed with brine (10 mL, dried with anhydrous MgSO₄ and evaporated under the reduced pressure. Trituration with benzene removed the unreacted starting steroide II and the remaining product was subsequently crystallized from CHCl₃: n-heptane (1:1) to give needle-like crystals (134 mg; 89%).

[α]_{D}=+88.4 (c 0.243);

¹H NMR (500 MHz, CDCl₃) δ 4.76-4.68 (m, 1H, 3-CH), 3.73-3.73 (bm, 2H, 4′-CH₂), 3.47 (s, 9H, NCH₃), 2.55 (t, 1H, J=9.0 Hz, 17-CH), 2.49 (t, 2H, J=6.2 Hz, 2′-CH₂), 2.12 (s, 3H, 21-CH₃), 0.94 (s, 3H, 19-CH₃), 0.60 (s, 3H, 18-CH₃).

¹³C NMR (101 MHz, CDCl₃) δ 209.47, 171.49, 75.20, 65.61, 63.79, 56.62, 53.45, 44.26, 41.83, 40.41, 39.13, 35.76, 34.96, 34.59, 32.19, 31.46, 30.27, 26.87, 26.59, 26.24, 24.37, 23.22, 22.89, 20.82, 18.46, 13.38.

IR (CHCl₃): 2956 (NMe₃⁺), 1722 (C═O, ester), 1699 (C═O, ketone), 1478 (NMe₃⁺) 1386 (CH₃), 1360 (COCH₃), 1230 (NMe₃⁺), 1188 (CO), cm⁻¹.

ESI m/z 446.6 (100%, [M−Cl]⁺); HRMS-ESI m/z 446.3624 ([M−Cl]⁺, C28H48O3N requires 446.3629).

For C28H48ClNO3 (482.1) calculated: 69.75%; C, 10.03%; H, 7.35%; Cl, 2.91%; N, found: 69.59%; C, 9.99%; H, 7.12%; Cl, 2.82% N.

### Example 7

### 20-Oxo-5β-pregnan-3α-yl 4-(trimethylammonium) hexanoate chloride

From 5-carboxy-N,N,N-trimethylpropan-1-aminium chloride (210 mg, 1 mmol) by similar procedure as in example 6 was prepared 20-oxo-5β-pregnan-3α-yl-4-(trimethylamonium) hexanoate hydrochloride (101 mg; 76%).

m.p.=205.5-207.5° C. (decomposition)

[α]_{D}=+87.7 (c 0.204)

¹H NMR (500 MHz, CDCl₃) δ 4.74-4.67 (m, 1H, 3-CH), 3.71-3.53 (bm, 2H, 5′-CH₂), 3.45 (s, 9H, NCH₃), 2.56 (t, 1H, J=8.9 Hz, 17-CH), 2.49 (t, 2H, J=6.1 Hz, 2′-CH₂), 2.12 (s, 3H, 21-CH₃), 0.95 (s, 3H, 19-CH₃), 0.61 (s, 3H, 18-CH₃).

¹³C NMR (101 MHz, CDCl₃) δ 209.60, 172.65, 74.30, 66.50, 63.78, 56.59, 53.27, 44.26, 41.77, 40.33, 39.11, 35.72, 34.96, 34.57, 34.08, 32.20, 31.50, 26.85, 26.62, 26.24, 25.64, 24.36, 24.32, 23.23, 22.89, 22.82, 20.78, 13.37.

IR (CHCl₃): 2958 (NMe₃⁺), 1720 (C═O, ester), 1700 (C═O, keton), 1478 (NMe₃⁺) 1386 (CH₃), 1360 (COCH₃), 1231 (NMe₃⁺), 1187 (CO), cm⁻¹.

ESI m/z 474.4 (100%, [M−Cl]⁺); HRMS-ESI m/z 474.3949 ([M−Cl]⁺, for C₂₈H₄₈O₃N calculated 474.3947).

### Example 8

### 20-Oxo-5β-pregnan-3α-yl-2-(trimethylammonium) acetate hydrochloride

Betain chloride hydrochloride was prepared from betaine (62 mg, 0.4 mmol) in dry dichloromethane (1 ml) under argon atmosphere. To the reaction mixture cooled in ice bath oxalyl chloride was added dropwise (0.5 ml; 5.82 mmol) followed by addition of catalytic amount of anhydrous dimethylformamide (3 μl; 0.03 mmol). Heterogenous mixture was let to reach room temperature and stirred for 16 h. During that time all solids were dissolved. Then the solvents were evaporated under reduced pressure and solid was dissolved in nitromethane (2 ml) and dry pyridine (3 μl; 0.03 mmol) in argon atmosphere. To the solution of chloride the compound II was added (100 mg; 0.31 mmol). Reaction mixture was stirred 4 h. The reaction was quenched by addition of water (10 ml). The mixture was acidified to pH 4 with 5% aq. HCl. Product was extracted with CHCl₃ (3×20 mL). Combined organic phase was dried with anhydrous MgSO₄ and evaporated under reduced pressure. Trituration with benzene removed the unreacted lipophilic starting material and the remaining product was subsequently crystallized from CHCl₃: n-heptan, 1:1 to give white crystals (81 mg; 62%).

m.p.=225-226° C. (decomposition)

[α]_{D}=+97.8 (c 0.231)

¹H NMR (500 MHz, CDCl₃) δ 4.76-4.68 (m, 1H, 3-CH), 3.78 (d, 2H, 2′-CH₂), 3.49 (s, 9H, NCH₃), 2.53 (t, 1H, J=9.0 Hz, 17-CH), 2.12 (s, 3H, 21-CH₃), 0.94 (s, 3H, 19-CH₃), 0.60 (s, 3H, 18-CH₃).

¹³C NMR (101 MHz, CDCl₃) δ 209.53, 164.26, 77.75, 63.76, 63.28, 56.50, 54.22, 44.26, 41.84, 40.34, 39.04, 35.74, 34.85, 34.56, 31.92, 31.50, 26.80, 26.36, 26.15, 24.37, 23.13, 22.90, 20.83, 13.39.

IR (CHCl₃): 2956 (NMe₃⁺), 1722 (C═O, ester), 1699 (C═O, keton), 1478 (NMe₃⁺) 1386 (CH₃), 1360 (COCH₃), 1230 (NMe₃⁺), 1188 (CO), cm⁻¹.

ESI m/z 418.3 (100%, [M−Cl]⁺); HRMS-ESI m/z 418.3323 ([M−Cl]⁺, pro C₂₈H₄₈O₃N vypo\check{c}teno 446.3319).

### Example 9

### Newly Added

### 3α-Amino-5β-pregnan-20-one hydrochloride salt

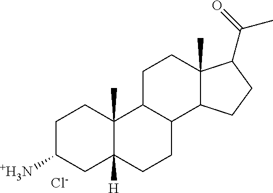 [SMILES: CC(=O)C1CCC2C3CC[C@@H]4C[C@H]([NH3+])CC[C@]4(C)C3CC[C@]12C ; [Cl-]]

3-α-Azido-5β-pregnan-20-one (2.98 g; 8.68 mmol) was dissolved in MeOH (150 mL), 5% Pd/CaCO₃ catalyst was added to the solution and the steroid was hydrogenated for 3 hrs under a slight overpressure of hydrogen. After the conversion was complete, the catalyst was filtered off, the solvent was partially removed in vacuo, the residue was poured into 5% aq. HCl (400 mL) and extracted with CHCl₃ (5×50 mL). The organic phase was dried with Na₂SO₄ and evaporated on rotavap. Crystallization of the oily residue from CHCl₃:n-heptane afforded white crystals of salt 3α-amino-5β-pregnan-20-one hydrochloride salt (2.73 g; 89%).

m.p.=276-277.5° C.

[α]_{D}=+103.1 (c 0.258).

¹H NMR (400 MHz, CDCl₃) δ 0.59 (s, 3H, 18-CH₃); 0.96 (s, 3H, 19-CH₃); 2.11 (s, 3H, 21-CH₃); 2.55 (t, 1H, J=8.9, 17-CH); 3.15-3.27 (bm, 1H, 3-CH); 8.33 (bs, 3H, NH₃⁺).

¹³C NMR (101 MHz, CDCl₃) δ 209.37, 63.68, 56.28, 51.92, 44.21, 42.17, 40.34, 38.93, 35.81, 35.15, 34.62, 31.60, 31.46, 26.73, 26.29, 26.18, 24.43, 23.33, 22.95, 20.89, 13.39.

IR (CHCl₃): 1157, 1193 (CO), 1238, 1255 (NMe₃⁺), 1359, 1386 (CH₃), 1451 (COCH₃), 1700 (C═O, ketone), 1721 (C═O, ester), 2958 (NMe₃⁺).

ESI m/z 318.4 (100%, [M]⁺), 340.4 (17%, [M−H+Na]⁺); HRMS-ESI m/z 318.27922 ([M]⁺, C₂₁H₃₆ON requires 318.27914).

### Example 10

### 3-(Trimethylammonium)-5β-pregnan-20-one chloride

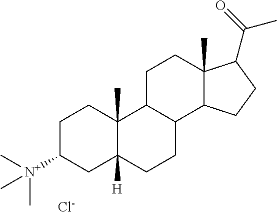 [SMILES: CC(=O)C1CCC2C3CC[C@@H]4C[C@H]([N+](C)(C)C)CC[C@]4(C)C3CC[C@]12C ; [Cl-]]

3α-Amino-5β-pregnan-20-one hydrochloride salt (200 mg; 0.57 mmol) was dissolved in MeOH (10 mL) and NaHCO₃ (285 mg; 3.39 mmol) was added to the solution. Finally, methyl iodide (0.22 mL; 3.53 mmol) was added to the stirred suspension and the mixture was refluxed for 3 days. The reaction mixture was then concentrated on rotavap, the reaction mixture was poured into 5% aqueous HCl (50 mL) and extracted with CHCl₃ (4×15 mL). The combined organic phase was washed with brine, dried over Na₂SO₄ and evaporated on rotavap. The solid residue was dissolved in minimal amount of MeOH and this solution was applied to a column of Amberlite IRA-400 (in Cl⁻ phase; 1×20 cm; packed in MeOH) and eluted slowly with the same solvent. The fraction containing steroid was collected, evaporated and crystallized from Et₂O to afford white crystals (151 mg; 67%).

m.p.=237-240° C. (decomposition);

[α]_{D}=+91.9 (c 0.235).

¹H NMR (1H, J=9.0, 17-CH); 3.42 (s, 9H, NCH₃) 3.66 (tt, 2H, ²J₁=12.1, ²J₂=3.3, 3-CH).

¹³C NMR (101 MHz, CDCl₃) δ 209.38, 75.46, 63.64, 56.25, 51.52, 44.18, 42.60, 40.74, 38.83, 35.71, 35.25, 34.41, 31.47, 27.00, 26.81, 26.24, 24.32, 22.95, 22.93, 21.39, 20.83, 13.40.

IR (CHCl₃): 2962 (NMe₃⁺), 1699 (C═O, ketone), 1488 (NMe₃⁺), 1386 (CH₃), 1359 (COCH₃).

ESI m/z 360.3 (100%, [M−Cl]⁺); HRMS-ESI m/z 360.32609 ([M−Cl]⁺, C₂₄H₄₂NO requires 360.32697).

### Example 11

### Effects of Pregnanolone Sulfate and its Cationic Analogs on Recombinant NMDA Receptors

HEK293 cells (American Type Culture Collection, ATTC No. CRL1573, Rockville, Md.) were cultivated in Opti-MEM® I media (Invitrogen) with addition of 5% fetal bovine serum at 37° C. and transfected with NR1-1a/NR2B/GFP plasmids, as described in the scientific literature (Cais et al., 2008). Same amounts (0.3 μg) of cDNA coding NR1, NR2 and GFP (green fluorescent protein) (pQBI 25, Takara, Japan) were mixed with 0.9 μl of Matra-A Reagent (IBA, Göttingen, Germany) and added to confluent HEK293 cells cultivated in v 24-pit cultivating plate. After trypsination, the cells were re-suspended in Opti-MEM® I containing 1% fetal bovine serum. Subsequently, 20 mmol·l⁻¹ MgCl₂, 1 mmol D,L-2-amino-5-phosphonopentanoic acid, 3 mmol·l⁻¹ kynurenic acid was added to the mixture and cells were inoculated on the polylysine-coated glass plates having 25 mm in diameter. The following genes coding NMDA receptor subunits were used for transfection: NR1-1a (GenBank accession no. U08261) and NR2B (GenBank accession no. M91562).

HEK293 Cultured cells were used for electrophysiological investigations with a latency of 16-40 h after transfection. Whole-cell currents were measured by patch-clamp amplifier (Axopatch 1D; Axon Instruments, Inc. Foster City, USA) after capacitance and serial resistance (<10 MΩ) compensation to 80-90%. Agonist-induced responses were filtered to 1 kHz (8-pole Bessel filter; Frequency Devices, Haverhill, USA), digitized with sampling frequency of 5 kHz and analyzed by pClamp version 9 software (Axon Instruments, USA). Micropipettes made of borosilicate glass were filled with intracellular solution, containing 125 mmol·l⁻¹ D-glukonic acid, 15 mmol·l⁻¹ cesium chloride, 5 mmol·l⁻¹ EGTA, 10 mmol·l⁻¹ HEPES buffer, 3 mmol·l⁻¹ magnesium chloride, 0.5 mmol·l⁻¹ calcium chloride and 2 mmol·l⁻¹ magnesium-salt of ATP (pH adjusted to 7.2 by cesium hydroxide solution). Extracellular solution (ECS) contained 160 mmol·l⁻¹ sodium chloride, 2.5 mmol·l⁻¹ potassium chloride, 10 mmol·l⁻¹ HEPES, 10 mmol·l⁻¹ glucose, 0.2 mmol·l⁻¹ EDTA a 0.7 mmol·l⁻¹ calcium chloride (pH adjusted to 7.3 by sodium hydroxide solution). Glycine was added to both testing and control solution. Moreover, bicuculline (10 gmol·l⁻¹) and tetrodotoxin (0.5 μmol·l⁻¹) was added to hippocampal cultures. Steroid-containing solutions were prepared from fresh solution (20 mmol·l⁻¹) of steroid dissolved in dimethyl-sulfoxide (DMSO). Same concentrations of DMSO were used in all extracellular solutions. Control and experimental solutions were applied via microprocessor-controlled perfusion system with approx. rate of solution exchange in areas adjacent to cells reaching ˜10 ms.

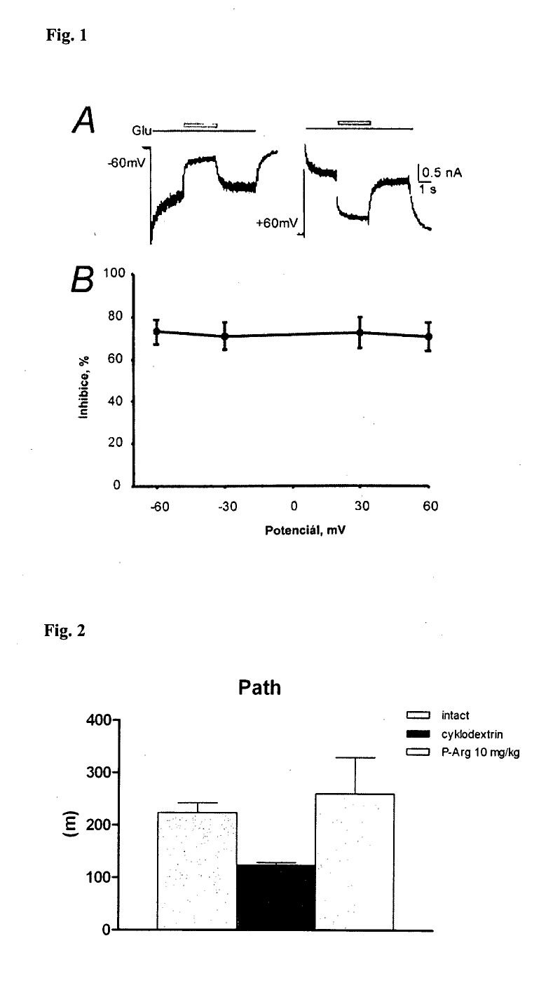

Current responses produced by 100 mmol·l⁻¹ of NMDA (in the case of hipocampal neurones), or by 1 mmol·l⁻¹ of glutamate (on recombinant NMDA receptors) were measured at membrane potential maintained at −60 mV. Similarly as described before, pregnanolone sulfate decreased the amplitude of responses elicited by NMDA. After application of 100 μmol·l⁻¹ of pregnanolone sulfate the mean inhibition effect reached 71.3±5.0 (n=5) on hipocampal neurones, and 67.2±8.2% (n=5) on recombinant NR1/NR2B receptors (Petrovic et al., 2005, 25(37), 8439-50). Our synthetic analogs of pregnanolone sulfate exhibited significant inhibitory effect (FIG. 1) at concentrations 50, 100, or 200 μmmol·l⁻¹ (chosen to reach maximal inhibition from 30 to 70%). Relative effect of steroid-induced inhibition was used for calculating IC₅₀. IC₅₀ was calculated using formula RI=1−(1/1+([steroid]/IC₅₀)^{h}), where RI denotes relative effect of steroid-induced inhibition and h is a parameter of Hill's coefficient (1.2). IC₅₀ values are stated in the following table.

Newly synthesized analogs from Examples 1-6 have the same mechanism of action on the NMDA receptors as pregnanolone sulfate, but they differ in their relative affinities (see Table 1).

TABLE 1

| Tested compound: | Relative inhibition | | Number | Concentration |
|---|---|---|---|---|
| Compound from example | effect (%) | IC₅₀ (μmol) | of cells | (μmol · l⁻¹) |
| Pregnanolone sulfate | 67.2 ± 8.2 | 55 | 5 | 100 |
| Compound from example 2 | 69.2 ± 9.6 | 5.3 +/− 2.1 | 5 | 10 |
| Compound from example 6 | 65.4 ± 3.4 | 5.9 +/− 0.7 | 5 | 10 |
| Compound from example 5 | 79.2 ± 4.2 | 3.3 ± 0.7 | 5 | 10 |
| Compound from example 7 | 85.6 +/− 2.3 | 23.6 +/− 3.5 | 5 | 100 |
| Compound from example 8 | 57.0 ± 9.6 | 22.4 +/− 4.1 | 5 | 30 |
| Compound from example 10 | 69.2 ± 9.6 | 106.7 +/− 12.1 | 5 | 100 |

The results show that synthetic analogs of pregnanolone sulfate has the same mechanism of action on NMDA receptors as pregnanolone sulfate; however, they differ in their affinity for these receptors. All experiments complied with standard accepted rules for animal care (Animal Protection Code of the Czech Republic, EU directives, and National Institute of Health guidelines).

### Example 12

### Effect of Compound from Example 2 (Pregnanolone Argininate) on Spontaneous Locomotor Activity in the Open-Field Test

For assessment of the spontaneous locomotor activity, animals (mice strain B6) were observed for 60 min in a circular open-field apparatus (diameter 82 cm). Animals were tracked with iTrack (Biosignal group, USA) and total path during this session was evaluated. The pregnanolone argininate was applied subcutaneously (dissolved in cyclodextrin) prior to behavioral observations at doses 10 mg/kg; control animals were injected with saline and cyclodextrin.

Results showed that pregnanolone argininate did not significantly alter the spontaneous locomotor activity. Both control groups exhibited similar total distance though there is apparent, yet insignificant, tendency of cyclodextrin to suppress locomotion. (see FIG. 2).

### Example 13

### Effect of Compound from Example 2 (Pregnanolone Argininate) and Pregnanolone Nitrate on Locomotion and Cognitive Function in Carousel Maze

Effect of pregnanolone argininate and nitrate and action of dizocilpine (a non-competitive antagonist of NMDA receptors; standardly used as positive control and referred hereafter as dizocilpine) on the behavior of rats in Carousel maze (active allothetic place avoidance (AAPA)), a spatial task requiring spatial orientation and cognitive functions

The effect of compounds on cognitive behaviors and locomotion was assessed using Carousel maze (active allothetic place avoidance (AAPA) task), which requires intact hippocampi and ability of animals to navigate in space using distinct reference frames (spatial navigation and “cognitive coordination”) (Wesierska et al., 2005) and it also allows measuring changes in the accompanying locomotor activity. AAPA task training involves animals trained to move over a slowly rotating uniform circular arena, on which a prohibited sector is defined, entering which is punished by a mild footshock. A shock sector and its position can be determined solely by its relationship to distal orienting cues in the room (Stuchlik et al., 2008).

This task is highly dependent upon hippocampal formation, with unilateral reversible ablation of this structure with TTX leading to avoidance deficit (Cimadevilla et al., 2001). Animals solving this task should walk in a direction opposite to arena rotation; otherwise it would be repeatedly brought to a fixed sector by arena rotation. We conducted 4 acquisition session of AAPA task, during which the sector location was always reinforced and remained stable throughout the training. Each session lasted 20 min.

The effect was tested on 2-month-old Long-Evans male rats. Control animals obtained cyklodextrine (s.c.), experimental groups received subcutaneous (s.c.) injections of the compound from example 2 at doses 1 mg/kg (dissolved in cyclodextrin). Intraperitonal application of dizocilpine (a non-competitive NMDA receptor antagonist; showing significant neuroprotective activity but exerting cognition-disturbing and psychotomimetic effects) was used as a positive control. Dizocilpine was applied at doses 0.15 mg/kg. It is worth noting that dizocilpine (albeit its experimental neuroprotective activity) is often used to model schizophrenia-like behaviors in humans and it has been repeatedly shown to exert a dose-dependent learning deficit and hyperlocomotion.

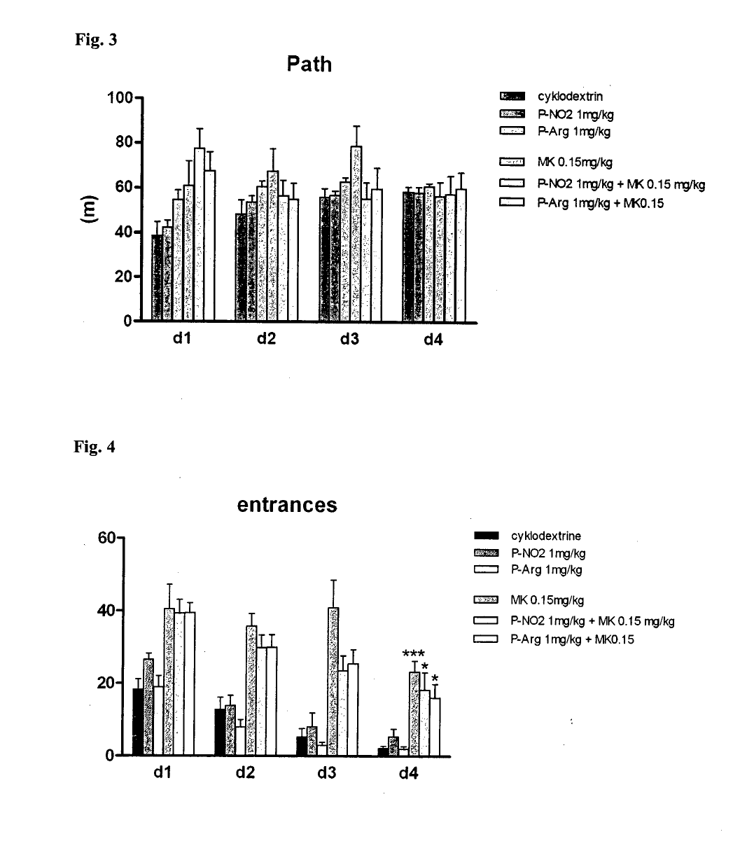

The results in four daily sessions are shown in FIG. 3. For clarity, statistical evaluations were performed for the final sessions (representing asymptotic levels of control animals); this approach has repeatedly proved useful in evaluation on neuropharmacological data from. AAPA task.

Locomotor activity of animals was assessed in the AAPA as total distance traveled in the coordinate frame of the arena (without passive rotation) in each 20-min session. Animals treated with pregnanolone argininate and nitrate failed to exhibit either decrease or increase in total distance compared to controls, whilst dizocilpine stimulated the locomotor activity.

It should be emphasized that hyperactivity (hyperlocomotion) induced by antagonists of NMDA receptors is sometimes considered to be into certain extent analogous to human positive symptoms of schizophrenia as both phenomena has been shown to relate to hyperfunction of mesolimbic dopaminergic circuits. Evaluation of locomotor activity in the AAPA task has shown that pregnanolone argininate and nitrate did not alter locomotor activity in this behavioral configuration. The results of activity analysis are depicted in FIG. 3.

FIG. 3. shows the total distance per session in the AAPA training, after application dizocilpine and pregnanolone argininate and nitrate. Neither drug caused significant alteration of locomotor activity when evaluated on session 4.

Number of Entrances into Shock Sector

Another parameter, which can be measured and which brings a spatial aspect to behavioral analysis, is number of entrances into prohibited sector (occasionally termed “number of errors”). This parameter actually shows the overall spatial performance of the task, indicating “efficiency, in which can rats learn this task”, and it is related to cognitive functions of animals. Results have shown that pregnanolone argininate and nitrate do not caused worsening in this parameter, whilst dizocilpine dose-dependently disrupted this parameter. Results of this experiment are shown in FIG. 4.

FIG. 4 illustrates the number of entrances (as a measure of cognitive functions) in every AAPA session after application of dizocilpine and pregnanolone argininate and nitrate. The dizocilpine led to impairment in this parameter whilst pregnanolone argininate and nitrate did not cause a statistical change in this parameter in comparison with controls. * denotes p<0.05, *** denotes p<0.001 compared to cyclodextrin controls; statistical test performed for the final session (see above).

Taken together the results show no significant effect of compound from example 2 on locomotion activity and spatial cognition. On the other side MK-801, noncompetitive NMDA antagonist, disrupts a spatial cognition. Absence of serious side effect (typical for NMDA antagonists) of pregnanolone argininate and nitrate can be cleared up by mechanism of action (use-dependent action).

### Example 14

### Anesthetic Effect of Compound from Example 2 (Pregnanolone Argininate)

Anesthetic effect of 3α5β pregnanolon argininate was tested on 2-month-old male mice strain B6. Animals were randomly assigned to 3 groups per 7 animals each. The first group was injected i.p. with saline, the second group received i.p. cyclodextrine, the third group obtained received the pregnanolon argininate at a dose 100 mg/kg (dissolved in β-cyclodextrine).

Application of pregnanolon argininate lead to decreased locomotor activity (3-15 minutes after injection) decreased locomotor activity. Decreased locomotor activity gradually changed into anesthesia of animals (5-20 minutes after injection). Animals spontaneously awoke after 1-3 hours. Anesthetic effect related to the mechanism of action of this compound. Animals in controls groups did not display any behavioral and locomotion changes.

### INDUSTRIAL APPLICABILITY

The compounds mentioned in the presented patent are industrially producible and applicable for treatment of numerous diseases of central nervous system, e.g. the following:

1) hypoxic and ischemic damage of the central nervous system, stroke, and other excitotoxicity-induced pathological alterations.
2) neurodegenerative changes and disorders
3) affective disorders, depression, PTSD and other stress-related diseases
4) schizophrenia and other psychotic disorders
5) pain, hyperalgesia and disorders of nociception
6) addictive disorders
7) multiple sclerosis and other autoimmune diseases
8) epilepsy and other seizure disorders
9) hyperplasic changes in CNS, CNS tumors including gliomas

### LITERATURE CITED

1. Cais O., Sedlá\check{c}ek M., Horak J., Dittert I., Vyklický, ml. L. Temperature dependence of NR1/NR2B NMDA receptor channels. Neuroscience 2008, 151(2), 428-438.
2. Cimadevilla J M, Wesierska M, Fenton A A, Bures J; Inactivating one hippocampus impairs avoidance of a stable room-defined place during dissociation of arena cues from room cues by rotation of the arena. Proc. Natl. Acad. Sci. USA 98 (2001), 3531-3536.
3. Lindstedt G., Lindstedt S.; Studies on biosynhesis of carnitine. J. Biol. Chem. 1965, 240(1), 316-321.
4. Morrow A L.; Recent developments in the significance and therapeutic relevance of neuroactive steroids-Introduction to the special issue. Pharmacol. Ther. 2007, 116(1), 1-6.
5. Petrovic M., Sedlacek M., Horak M., Chodounska H., Vyklický L. Jr.; 20-oxo-5beta-pregnan-3alpha-yl is a use-dependent NMDA receptor inhibitor. J. Neurosci. 2005, 25(37), 8439-50.
6. Rambousek L., Bubenikova-Valesova V., Kacer P., Syslova K., Kenney J., Holubova K., Najmanova V., Zach P., Svoboda J., Stuchlik A., Chodounska H., Kapras V., Adamusova E., Borovska J., Vyklicky L., Vales K.; Cellular and behavioural effects of a new steroidal inhibitor of the N-methyl-D-aspartate receptor 3α5β-pregnanolone glutamate. Accepted for publication in Br. J. Pharmacol., DOI: 10.1111/j.1476-5381.2011.01816.x
7. Stuchlik A, Petrasek T, Vales K; Dopamine D2 receptors and alpha1-adrenoceptors synergistically modulate locomotion and behavior of rats in a place avoidance task. Behay. Brain Res. 189 (2008), 139-144.
8. Wesierska M; Dockery C; Fenton A A; Beyond memory, navigation, and inhibition: behavioral evidence for hippocampus-dependent cognitive coordination in the rat. J. Neurosci. 25(2005), 2413-2419.
9. Villmann C., Becker C M.; On the hypes and falls in neuroprotection: targeting the NMDA receptor. Neuroscientist. 2007, 13(6), 594-615.2.
10. Weaver C E., Land M B., Purdy R H., Ruchards K G., Gibbs T T., Farb D H.; J. Pharm. Exp. Ther. 293 (2000), 747.

1. Pregnanolone derivatives, substituted in the 3alpha-position with the cationic group, of general formula I 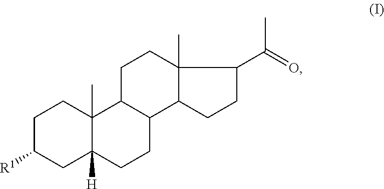
wherein R¹ represents the group of general formula R³—R²—C(R¹³)—R⁴—,
where R² means (Cₘ)ₙ-group wherein m is 0 to 2, n is 1 to 18 which forms straight or a branched chain, which may be additionally substituted by one or more halogen atoms or primary, secondary or terciary amino group, which may be either free or in case of primary amino group protected by a removable protecting group, chosen from tert-butoxycarbonyl, trityl, benzyloxycarbonyl, 9-fluorenylmethoxycarbonyl or p-nitrobenzyloxycarbonyl, R³ represents cationic group, chosen from guanidyl group of general formula (a) 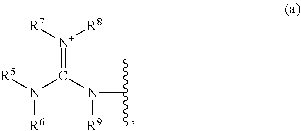
or ammonium group of general formula (b) 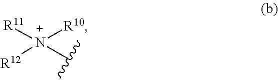
where R⁵-R¹² are hydrogen atoms or alkyl or alkenyl groups with 1 to 18 carbon atoms in a straight or a branched carbon chain, R¹³ is chosen from a group, involving oxygen, nitrogen or sulfur atom bound by a double bond to carbon, or R¹³ are two hydrogen atoms; R⁴ is bivalent or multivalent atom, chosen from oxygen, nitrogen or carbon atom and in case where R⁴ is multivalent atom, that is carbon or nitrogen, its additional valent or valents are one or more hydrogens and any of hydrogen atoms may be substituted by alkyl or alkenyl having from 1 to 4 carbon atoms.

2. Method of production of pregnanolone derivatives, substituted in 3 alpha-position by cationic group, of general formula I according to claim 1, where R¹ is as given above and the substituent R³ is guanidyl group of the formula (a) and R⁴ means oxygen atom wherein the reacti y-5beta-pregnane-20-one of formula II 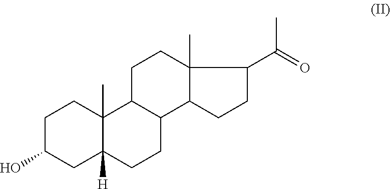
and arginine protected by suitable protecting group chosen from tosyl, 2,2,5,7,8-pentamethyl-6-sulfonyl, 2,2,4,6,7-pentamethyl-di hydrobenzofuran-5-sulfonyl, mesityl-2-sulfonyl, 4-methoxy-2,3,6-trimethylphenylsulfonyl, 1,2-dimethylindole-3-sulfonyl, ω,ω′-bis-tert-butyloxycarbonyl, w-nitro, trifluoroacetyl, ω,ω′-bis-benzyloxycarbonyl or ω,ω′-bis-allyloxycarbonyl group is dissolved in suitable dry solvent, chosen from chloroform, dichloromethane, benzene, toluene, ethylacetate, or acetonitrile under the inert atmosphere, the reaction mixture is then cooled in ice bath, condensing agents, which is dicyclohexylcarbodiimide or 1-(3-dimethylamino-propyl)-3-ethylcarbodiimide and a catalytic agent being dimethylaminopyridine, dissolved in convenient solvent chosen from benzene or toluene, are added dropwise to the stirred mixture; the reaction mixture is prevented against the air humidity and stirred 10-48 hours at temperatures between 0 and 50° C., then is poured into saturated water solution of sodium or potassium bicarbonate and the product is extracted with an organic solvent, in which is well soluble, collected organic phases are then washed with saturated water solution of sodium chloride until sodium bicarbonate is removed, the extract is dried over magnesium sulfate or sodium sulfate and the solvent is evaporated preferably by distillation under vacuum; the crude material is triturated with minimal amount of acetone and precipitated dicyclohexylurea is filtered off to obtain the compound of general formula I which can be then purified where appropriate and in the process a possible protecting group of arginine moiety is removed so that the obtained compound is dissolved in a mixture of carboxylic acid and alcohol, to this solution a hydrogenation catalyst is added, preferably Pd/C or platinum black and after the hydrogenation during 48-72 h the catalyst is filtered off and the solvent is evaporated.

3. Method of production according to claim 2, wherein the reaction mixture is mixed from 10 to 12 h, acetonitrile is used as organic solvent, the product is purified by crystallization, or by chromatography on the silica gel column and in case of removing a protecting group preferably the mixture of acetic acid and methanol is used and the time of hydrogenation is preferably 72 hours.

4. Method of production according to claim 2, wherein the protecting group of arginine structure of the compound of general formula I, being benzyloxycarbonyl group or (2,2,4,6,7-pentamethyl-dihydrobenzofuran-5-sulfonyl) group, is removed by trifluoroacetic acid treatment of the protected derivative; the reaction mixture is allowed to react from 16 to 72 hours at temperatures from 0° to 50° C., then the mixture is poured into saturated solution of sodium or potassium bicarbonate and the product is extracted with an organic solvent in which is well soluble, chosen from a group, including chloroform, dichloromethane or dichloroethane; collected organic phases are washed with 5% aqueous hydrochloric acid, the extract is dried over drying agent and the solvent is evaporated after which the crude material is purified where appropriate, e.g. by crystallization to afford the dihydrochloride of compound of general formula I.

5. Method of production according to claim 4 wherein the reaction mixture is allowed to react under the room temperature, the bicarbonate used is sodium bicarbonate, drying agent is magnesium or sodium sulfate and the solvent is preferably evaporated by distillation under the vacuum.

6. The method of the preparation of compound of general formula I mentioned in claim 1, where R¹ means as given above and substituent R³ is quaternary ammonium salt of formula (b) 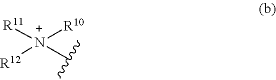
with the various length of chains connecting the amino-group with carboxyl creating ester bond with the compound of general formula II, wherein the appropriate quaternary salt of ω-carboxylic acid is suspended in anhydrous dichloromethane under inert atmosphere, suitable chlorinating agent, chosen from a group consisting of thionyl chloride, phosphorus oxychloride and oxalyl dichloride is added into the reaction mixture of the appropriate temperature from −50 to +20 deg C. and the reaction can be catalyzed by convenient catalyst; reaction mixture is then stirred for 8-72 h to dissolve all solids, volatile components are then evaporated in vacuum and crude product is dissolved in the mixture of dry nitromethane and pyridine under the inert atmosphere, afterwards the compound of general formula II is added and the mixture is then stirred for 2-24 until reaction is quenched with water, then acidified to pH 4.0 with 5% aqueous solution of hydrochloric acid, organic components are extracted into chloroform and this is washed with saturated solution of sodium chloride, obtained solution is dried over drying agent, solvent then evaporated and unreacted starting material is triturated with benzene to remove it; remaining product is purified if appropriate, e.g. by crystallization from the mixture of suitable solvents to give the product of general formula I, where R³ means as given in general formula (b).

7. The method of the preparation according to claim 6, characterized in that the temperature of the reaction mixture is preferably 0 deg C., as the chlorinating agent is preferably used oxalyldichloride and as the catalyst dimethylformamide, at the same time the reaction mixture is stirred at first 16 h and after the addition of the compound of general formula II next 4 hours, magnesium or sodium sulfate is used as drying agent, solvent is evaporated by distillation under the vacuum and the product is purified by crystallization from the mixture of chloroform and n-heptane.

8. Pregnanolone derivatives, substituted in 3 alpha-position with cationic group, of general formula I according to claim 1, for usage in treatment of neuropsychiatric disorders related to dysbalances of glutamatergic neurotransmitter system, as ischemic CNS injury, neurodegenerative changes, and disorders of central nervous system, mood disorders, depression, post-traumatic stress disorder, and other stress-related disorders, anxiety, schizophrenia, and other psychotic illnesses, pain, addiction, multiple sclerosis, epilepsy, and gliomas.

9. The use of pregnanolone derivatives, substituted in 3 alpha-position with cationic group, of general formula I according to claim 1 for production of pharmaceutical preparation for treatment of neuropsychiatric disorders related to imbalance of glutamatergic neurotransmitter system, such as ischemic damage of central nervous system, neurodegenerative changes and disorders of central nervous system, affective disorders, depression, post traumatic stress disorder, and other diseases related to stress, anxiety, schizophrenia, and psychotic disorders, pain, addictions, multiple sclerosis, epilepsy, and gliomas.

10. The use of pregnanolone derivatives, substituted in 3 alpha-position by cationic group, of general formula I according to claim 1 for production of veterinary and human pharmaceutical preparations for treatment of neuropsychiatric disorders related to imbalance of glutamatergic neurotransmitter system, such as ischemic damage of central nervous system, neurodegenerative changes and disorders of central nervous system, affective disorders, depression, post traumatic stress disorder, and other diseases related to stress, anxiety, schizophrenia, and psychotic disorders, pain, addictions, multiple sclerosis, epilepsy, and gliomas.

11. Pharmaceutical preparation containing pregnanolone derivatives, substituted in 3 alpha-position with cationic group, of general formula I as active component.

12. Pharmaceutical preparation according to claim 11 for treatment of neuropsychiatric disorders related to dysbalance of glutamatergic neurotransmitter system, especially ischemic central nervous system injury, neurodegenerative changes and disorders of central nervous system, mood disorders, depression, post-traumatic stress disorder, and other stress-related disorders, anxiety, schizophrenia, and other psychotic illnesses, pain, addiction, multiple sclerosis, epilepsy, and gliomas.

13. The usage of pregnanolone derivatives, substituted in 3 alpha-position with cationic group, of general formula I according the claim 1, for production of standards of neuroprotectives and neuroleptics, or analytical standards utilized in experimental research, analytic chemistry; also utilization of these compounds as active substances contained in dietary supplements or compounds contained in food supplements or cosmetic preparations designated for improvement of reactions of particular parts of organisms on higher stress, namely oxidative, nutrient, or caused by free radicals, or by ageing.

14. Pregnanolone derivatives, substituted in the 3alpha-position with the cationic group, of general formula I described in claim 1, in which substiuent —R²—C(R¹³)—R¹⁴— is missing, R¹═R³=ammonium group of general formula (b) described in claim 1 and R-R¹² of this group are hydrogen atoms or alkyl or alkenyl groups with 1 to 18 carbon atoms in a straight or a branched carbon chain.
